(19) United States
(12) Patent Application Publication
(10) Pub. No.: US 2018/0265804 A1
(43) Pub. Date: Sep. 20, 2018

(54) ESSENTIAL OIL DERIVATIVES, THEIR PREPARATION AND USES

(71) Applicants: Giovanni GASPERONI, Isola Dovarese (IT); VOLARE & CONNECTING, LLC, St. Louis, MO (US)

(72) Inventors: Irene BASSANETTI, Calestano (IT); Mauro CARCELLI, Parma (IT); Giuliano LEONARDI, Isola Dovarese (IT); Paolo PELAGATTI, Parma (IT); Dominga ROGOLINO, Isola Dovarese (IT)

(73) Assignees: VOLARE & CONNECTING, LLC, St. Louis, MO (US); Giovanni GASPERONI, Isola Dovarese (IT)

(21) Appl. No.: 15/571,222

(22) PCT Filed: May 15, 2015
(86) PCT No.: PCT/IB2015/053591

§ 371 Date: Nov. 1, 2017

(51) Int. Cl. C11B 9/00; C07C 309/62; A23K 20/111; A23L 33/115

(52) U.S. Cl. CPC C11B 9/0011; A23L 33/115; A23K 20/111; C07C 309/62

(57) ABSTRACT

Compounds are described which are derivatives of essential oils, functionalized in order to improve their antibacterial, antiprotozoal and antiviral activity, reduce their volatility and modify their solubility in certain solvents, particularly water. Additionally, also the palatability has been significantly improved with respect to the starting essential oils. These compounds are designed 5 to be used as food ingredients in food products for nutritional purposes and extra-nutritional (as prebiotics) purposes, in both human and animal feed industries, even if these compounds can be successfully used also in cosmetics, detergents and for the control of the microbial load in general.

### FIELD OF THE INVENTION

The present invention concerns compounds which are derivatives of essential oils, functionalized in order to improve their antibacterial, antiprotozoal and antiviral activity, reduce their volatility and modify their solubility in certain solvents, particularly water. Additionally, also the palatability has been significantly improved with respect to the starting essential oils.

These compounds are designed to be used as food ingredients in food products for nutritional purposes and extra-nutritional (as prebiotics) purposes, in both human and animal feed industries, even if these compounds can be successfully used also in cosmetics, detergents and for the control of the microbial load in general.

### STATE OF THE ART

The essential oils and their volatile constituents have been used for ages to treat and prevent human diseases. Recently, the interest for such essential oils has increased especially in consideration of their anticancer activity and their beneficial role in cardiovascular failure problems (arteriosclerosis, thrombosis). Essential oils are also known for their antibacterial, antiviral and antioxidant activities.

For these reasons, even the feed industry has recently revalued the use of essential oils for reducing antibiotic treatments, and as growth promoters in animal livestock.

Essential oils are also believed to have an immunostimulatory and anti-inflammatory capacity that improves the general resistance to bacteria and viruses, as well as a best response of animals to exogenous stress factors.

The use of essential oils, however, is not free from technological and practical difficulties ascribable to their chemical nature and the complex physiology of the different animal species. The volatility, for instance, involves a rapid loss of the active ingredients during the production processes (dosing, mixing, hot pelleting, transporting and packaging), and also during storage and administration of feed, thus resulting in final amounts of essential oils unavoidably and uncontrollably reduced.

Additionally, the marked aromatic note of these oils, which is the most appreciable feature in applications such as aromatherapy or perfume production, is conversely one of the major limitations in food and feed applications, especially at therapeutic dosages, because the final products result to have an unpleasant taste, which discourages the consumption.

Currently, essential oils are coated or microencapsulated in order to mask the unpleasant taste, depending on the target area of the digestive tract (rumen, stomach, intestines . . . ) where they have to be released. However, this means that also their antibacterial and antioxidant effect towards the food containing the same is undesirably and inconveniently masked and nullified.

Therefore, it is an object to overcome the drawbacks of the essential oils, such as volatility e marked aromatic note, in order to take benefit of essential oil properties.

### SUMMARY OF THE INVENTION

The above object has been achieved by a compound of formula A-O—R, as described in claim 1.

In a further aspect, processes for producing said compound are provided.

In another aspect, a precursor for said processes is provided.

In an even further aspect, the present invention relates to the use of said compound of formula A-O—R or said precursor as a food ingredient in human and animal food products.

The characteristics and the advantages of the present invention will become apparent from the following detailed description and from the working examples provided for illustrative purposes.

### DETAILED DESCRIPTION OF THE INVENTION

The subject of the invention therefore is a compound of formula A-O—R, wherein A-O— is a moiety of an essential oil active ingredient, said active ingredient having a functional group —OH, and R is a moiety of formula R₁(CO)— or formula A-O—(CO)—R₂—(CO)—, where R₁ is a saturated or unsaturated alkyl chain C4-C22 and R₂ is a saturated or unsaturated alkyl chain C1-C6.

With “compound of formula A-O—R”, it is meant to include also salts, isomers, and racemic mixtures thereof.

It has been surprisingly found that the compounds of the invention are stable, not volatile at room temperature, and have a more acceptable and pleasant taste, so that the palatability has been significantly improved with respect to the starting essential oils.

Moreover, their antibacterial, antiprotozoal and antiviral activity has been observed and their solubility in certain solvents has been improved with respect to the starting essential oils.

Essential oils are typically concentrated, hydrophobic liquid matrices containing volatile aroma compounds from plants. Essential oils are generally extracted by distillation, often by using steam. Other processes include expression or solvent extraction. Essential oils may also be obtained through synthetic or semi-synthetic routes.

For the purposes of the present invention, A-O— is a moiety of an essential oil active ingredient, i.e. it is a volatile aroma compound present in essential oils, having a functional group —OH. In some embodiments, said active ingredient of essential oils is selected from the group consisting of menthol, thymol, eugenol, geraniol, vanillin, carvacrol, nerolidol, ethyl maltol, furaneol, 1-hexanol, 3-hexen-1-ol, linalool, alpha-santalol, nerol, terpineol, borneol, farnesol, citronellol, myrtenol, coumaric acid, para-hydroxybenzoic acid, gallic acid, protocatechuic acid, syringic acid, gentisic acid, caffeic acid, ferulic acid, sinapic acid, methyl salicylate, and mixtures thereof. Preferably, said active ingredient is selected from the group consisting of menthol, thymol, eugenol, vanillin, carvacrol, and mixtures thereof.

Preferably, said moiety R of formula R₁(CO)— is a moiety of myristoleic acid, palmitoleic acid, sapienic acid, oleic acid, elaidic acid, vaccenic acid, linoleic acid, linoelaidic acid, α-linolenic acid, arachidonic acid, eicosapentaenoic acid, erucic acid, docosahexaenoic acid, butyric acid, valeric acid, caproic acid, enanthic acid, caprylic acid, capric acid, lauric acid, myristic acid, palmitic acid, stearic acid, nonadecylic acid, arachidic acid, heneicosanic acid, or behenic acid. In preferred embodiments, said moiety R has formula R₁(CO)— and R₁ is a saturated alkyl chain C8-C18.

In other embodiments, said moiety R has formula A-O—(CO)—R₂—(CO)—, where —O—(CO)—R₂—(CO)— is a moiety of a dicarboxylic acid, such as oxalic acid, malonic acid, succinic acid, glutaric acid, adipic acid, maleic acid, fumaric acid, glutaconic acid, muconic acid, aspartic acid, glutamic acid, or tartaric acid.

In preferred embodiments, said moiety R has formula A-O—(CO)—R₂—(CO)— and R₂ is a saturated or mono-unsaturated alkyl chain C1-C4.

Particularly preferred are the following compounds of the invention:

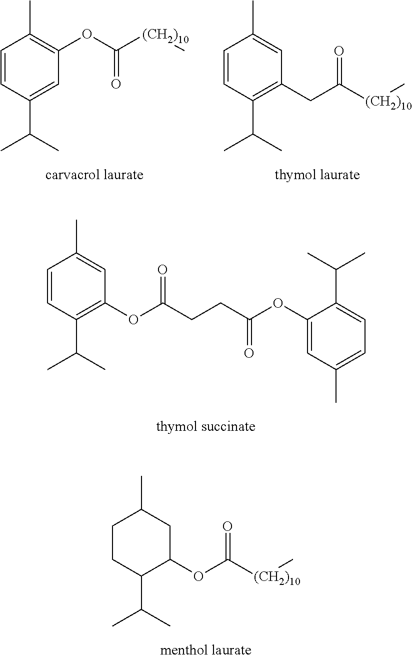

In another aspect, the present invention concerns a process for the preparation of the compound of formula A-O—R, wherein said moiety R has formula R₁(CO)—, said process comprising the steps of:

a) providing a carboxylic acid of formula R₁(CO)OH and an essential oil active ingredient of formula A-OH, being the carboxylic acid in excess over the active ingredient; and

b) heating up to melting of both the components of step a), under vacuum, under stirring and in the presence of an acid catalyst, and reacting for at least 9 hours.

This synthesis is an esterification between an alcohol group and a carboxyl group, but, in the classic Fisher esterification the alcohol is in excess, preferably in a stoichiometric excess, over the carboxylic acid, whereas in the synthesis of the present invention the carboxylic acid is in a stoichiometric excess over the alcohol.

The synthesis is advantageously solvent-free and makes use of an acid catalyst, such as H₃PO₄, under vacuum in order to remove water and thus promoting the reaction towards the formation of the final compound of formula A-O—R.

Preferably, in step b), the components react for at least 12 hours.

In a further aspect, the present invention concerns a process for the preparation of the compound of formula A-O—R, wherein said moiety R has formula A-O—(CO)—R₂—(CO)—, said process comprising the steps of:

a) reacting a dicarboxylic acid of formula HO(CO)—R₂—(CO)OH with oxalyl chloride under dry conditions, thus obtaining the respective acyl chloride; and

b) reacting said acyl chloride with an essential oil active ingredient of formula A-OH.

Preferably, a dry polar solvent is used in both step a) and step b), such as dry THF.

The process can be advantageously carried out at room temperature.

Preferably, in step b), the components react for at least 12 hours, and then the solvent is evaporated.

More preferably, the product resulting from step b) is washed by adding ethyl acetate and then water at least once. The resulting organic phase containing the compound of formula A-O—R is dried upon anhydrous Na₂SO₄, filtered and dried off.

In preferred embodiments of the processes above, said an essential oil active ingredient of formula A-OH is preliminarily reacted with sulfuric acid to give the respective sulfonate.

If A-OH is liquid at room temperature, it is not necessary to heat, it is only essential that A-OH is in a melted state so that the reaction does not require any solvent.

Preferably, sulfuric acid (98%) is added in excess (about 1 ml per 1 g) under stirring then applying a mild vacuum.

A deliquescent solid product can be obtained, still including an excess of sulfuric acid, which can be easily dissolved in an alcoholic solution, e.g. methanol solution, and then neutralized to neutral pH with a pH adjuster, such as an aqueous solution of a metal hydroxide, where the metal can be Ca²⁺, Li⁺, K⁺, Na⁺, Mg²⁺, or Cu⁺. The resulting metal sulfate can be filtered off and the remaining solution containing the sulfonate of essential oil active ingredient is let to dry.

This synthesis is thus solvent-free, very easy and rapid, has a very high yield of final product, almost quantitative, and does not require complex purification steps.

In another aspect, the present invention also concerns a sulfonate of essential oil active ingredient A-OH, or a salt thereof.

Preferably, said sulfonate of A-OH is sulfonate of menthol, thymol, eugenol, geraniol, vanillin, carvacrol, nerolidol, ethyl maltol, furaneol, 1-hexanol, 3-hexen-1-ol, linalool, alpha-santalol, nerol, terpineol, borneol, farnesol, citronellol, myrtenol, coumaric acid, para-hydroxybenzoic acid, gallic acid, protocatechuic acid, syringic acid, gentisic acid, caffeic acid, ferulic acid, sinapic acid, or methyl salicylate, or a salt thereof.

Suitable counter-ions for the salt of sulfonate of A-OH can be sodium and potassium.

Particularly preferred are the following sulfonates of the invention:

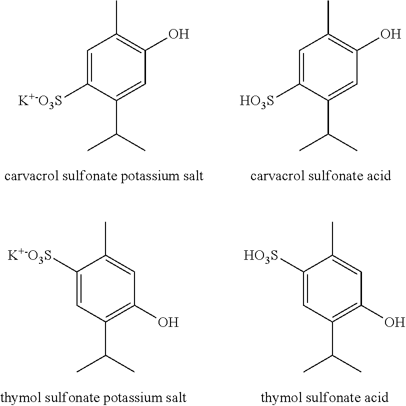

As will be apparent from the Examples below, the sulfonate of A-OH has been found to be advantageous in that it is a solid powder that can be easily handled and stored, has a good stability and a reduced aromatic note.

However, this compound it is not only a convenient intermediate material for the processes above described, but it also showed very remarkable properties, which allow to overcome the drawbacks of the essential oils, because it is not volatile, has a more pleasant taste, and interestingly has good solubility in water.

In a further aspect, the present invention concerns the use of the compound of formula A-O—R or the sulfonate of A-OH as a food ingredient in human and animal food products.

Indeed, these compounds can be used as effective and advantageous substitute of the respective essential oil active ingredient in all their applications, such as like antibacterial agent, antiviral agent and antioxidant agent.

With reference to the examples given below, the in vitro antibacterial activity of the compounds of the invention has been investigated. In this regard, without wishing to be bound by any theory, it is supposed that the lower activity shown by some esters in in vitro tests could be ascribable to the acid moiety, which is believed to act as a protective group. In fact, the esterification protects the essential oils throughout the production process and handling of the food products containing the same, up to the first digestion step. Then, once the intestine area is reached, the lipases intervene on the ester molecules which thus release the essential oils. In this way, the latter are free to exert there their antibacterial activity, i.e. exactly where pathogens such as E. coli and Salmonella are typically present.

The possibility to release essential oils once the esters of the invention contact lipases in the intestine area, i.e. the possibility to use the esters of the invention like pro-drug molecules, allows to achieve a number of advantages with respect to the essential oils as such:
higher yields during the production process due to the lower volatility of the esters,
less intense odour resulting in a higher palatability;
higher safety during the production process and handling due to the higher flash points of esters;
reduced reactivity towards other active principles present in food products, such as vitamins, metal oxides, etc . . . ;
combined action of the released essential oil and the carboxylic or dicarboxylic acid, the latter having itself antibacterial properties.

It has been observed also that essential oils and esters of the invention have different solubility in water and different melting points.

The remarkable solubility of the esters of the invention in water, compared to the insolubility of essential oils as such, is a matter of great interest. For example, these esters can be supplied also as beverage to animals. Indeed, by increasing the hydrophilicity of these products, also the bioavailability should improve.

Moreover, the higher melting points and the solid state (such as powders) instead of liquid oils ensure better handling in the production process, whereas higher flash points (lower flammability) result in more safety working conditions. Lower volatility is also an additional quality of the esters in terms of a reduced olfactory impact on animals during nourishment and a smaller loss ratio of active ingredient in manufacturing the food products.

It should be understood that all aspects identified as preferred and advantageous for the compound of formula A-O—R are to be deemed as similarly preferred and advantageous also for the sulfonate of A-OH, the respective processes of production, and uses of the present invention.

It should be also understood that all the combinations of preferred aspects of the compound of formula A-O—R, sulfonate of A-OH, their processes of production, as well as their uses, as above reported, are to be deemed as hereby disclosed.

Below are working examples of the present invention provided for illustrative purposes.

### EXAMPLES

### Example 1

### Carvacrol Sulfonic Acid (IB540)

Carvacrol (2.2 g, 0.013 mol) and sulfuric acid 96% w (2.1 ml, 0.038 mol) were mixed for 2 hours under vacuum at 30° C. The reaction was completed when the red sticky mixture became a pink solid.

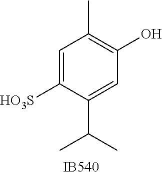

¹H-NMR (400 MHz, d⁶-DMSO, ppm, δ): 1.10 (d, 6H, 2CH₃(i-Pr)); 2.06 (s, 3H, CH₃); 3.94 (m, 1H, CH(i-Pr)); 6.73 (s, 1H, CH(Ph)); 7.45 (s, 1H, CH(Ph)).

### Example 2

### Carvacrol Sulfonate Potassium Salt (IB546)

Carvacrol sulfonic acid (5 g) was poured in 5 ml of methanol and neutralized (pH 7) with a saturated solution of KOH in water. The precipitate was removed by Buchner filtration and the filtrate was vacuum-dried yielding a white solid (3.3 g, 95%).

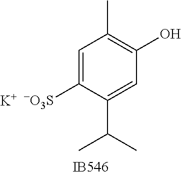

IR (cm⁻¹): 3213br, 2966w, 1853w, 1492w, 1410m, 1301w, 1272m, 1164m, 1134m, 1088m, 1042s, 976w, 887w, 717w, 665m, 609m, 580s.

¹H-NMR (400 MHz, d⁶-DMSO, ppm, δ): 1.09 (d, 6H, 2CH₃(i-Pr)); 2.04 (s, 3H, CH₃); 4.02 (m, 1H, CH(i-Pr)); 6.72 (s, 1H, CH(Ph)); 7.43 (s, 1H, CH(Ph)); 9.22 (s, 1H, OH).

MS (m/z, CI): 230.0 [C₁₀H₁₄O₄S]^{±}.

### Example 3

### Thymol Sulfonic Acid (IB574)

Thymol (2.2 g, 0.013 mol) and sulfuric acid 96% w (2.1 ml, 0.038 mol) were mixed for 2 hours under vacuum at 40° C. The reaction was completed when the yellow mixture became a pale pink solid.

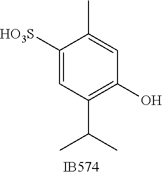

¹H-NMR (400 MHz, d⁶-DMSO, ppm, δ): 1.13 (d, 6H, 2CH₃(i-Pr)); 2.40 (s, 3H, CH₃); 3.14 (m, 1H, CH (i-Pr)); 6.56 (s, 1H, CH (Ph)); 7.51 (s, 1H, CH(Ph)).

### Example 4

### Thymol Sulfonate Potassium Salt (IB575)

Thymol sulfonic acid (5 g) was poured in 5 ml of methanol and neutralized (pH 7) with a saturated solution of KOH in water. The precipitate was removed by Buchner filtration and the filtrate was vacuum-dried yielding a white solid (3.2 g, 92%).

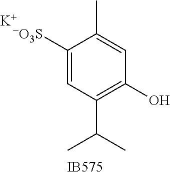

IR (cm⁻¹): 3415wbr, 2996w, 2871vw, 1611w, 1578w, 1493w, 1459m, 1403w, 1339w, 1259w, 1203m, 1158s, 1130m, 1105w, 1079m, 1038s, 903w, 883w, 867w, 733m, 664s.

¹H-NMR (400 MHz, d⁶-DMSO, ppm, δ): 1.99 (d, 6H, 2CH₃(i-Pr)); 2.38 (s, 3H, CH₃); 3.16 (m, 1H, CH(i-Pr)); 6.51 (s, 1H, CH(Ph)); 7.50 (s, 1H, CH(Ph)); 9.19 (s, 1H, OH).

MS (m/z, CI): 230.0 [C₁₀H₁₄O₄S]⁺.

### Example 5

### Thymol Succinate (IB559)

To a solution of succinic acid (1 g, 0.01 mol) in anhydrous THF (50 ml) with 3 drops of DMF as catalyst, oxalyl chloride (1.7 ml, 0.02 mol) was added in dry atmosphere and under magnetic stirring. One hour later, the volatilities were removed under vacuum, then freshly distilled THF (50 ml) was added and thymol (2.85 g, 0.02 mol) was poured in the mixture, and the reaction was stirred for 4 h. The volatilities were removed again and ethyl acetate (50 ml) was added. The organic phase was washed twice with water (50 ml) and with brine. Then it was dried with sodium sulfate and vacuum-dried. The product was a pale yellow oil (1.8 g, 52%).

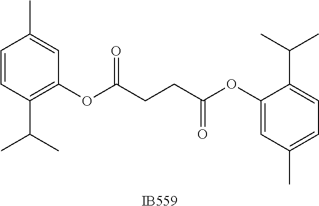 [SMILES: Cc1ccc(C(C)C)c(OC(=O)CCC(=O)Oc2cc(C)ccc2C(C)C)c1 ; "IB559"]

IR (cm⁻¹): 3429br, 2961s, 2921m, 2871w, 1709s, 1619m, 1584m, 1518w, 1458m, 1419s, 1375m, 1336w, 1289s, 1259s, 1227s, 1152s, 1112w, 1087m, 1043m, 1005w, 945m, 855w, 807s, 738m.

¹H-NMR (400 MHz, CDCl₃, ppm, δ): 1.26 (d, 12H, 4CH₃(i-Pr)); 2.07 (s, 2H, 2CH₂); 2.30 (s, 6H, 2CH₃); 3.19 (m, 2H, CH₂); 6.60 (s, 2H, 2CH (Ph)); 6.75 (d, 2H, J=7.6 Hz, 2CH(Ph)); 7.10 (d, 2H, J=7.6 Hz, 2CH(Ph)).

¹³C-NMR (400 MHz, CDCl₃, ppm, δ): 20.87 (CH₃); 23.05 (CH₃(i-Pr)); 29.06 (CH₂); 116.03 (CH(Ph)); 121.59 (CH(Ph)); 126.23 (CH(Ph)); 131.48 (CH(Ph)); 136.56 (CH(Ph)); 116.03 (CH(Ph)); 152.62 (CO).

ESI-MS (m/z): 405 [C₂₄H₃₀O₄Na]⁺; 363 [C₂₁H₂₄O₄Na]⁺; 273 [C₁₄H₁₈O₄Na]⁺.

### Example 6

### Carvacrol Laurate (IB536)

Carvacrol (2 g, 0.013 mol), lauric acid (2.6 g, 0.013 mol) and an 85% aqueous solution of phosphoric acid (3 drops) were mixed under magnetic stirring at 150° C. under vacuum for 12 hours. The crude was poured in chloroform (4 ml) and purified with a flash chromatographic column (silica gel, n-hexane:dichloromethane 8:2), the product remained in column, then pure ethyl acetate was added thus yielding a colourless oil (2.2 g, 51%).

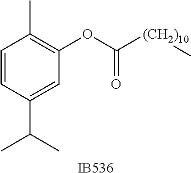

IR (cm⁻¹): 2954m, 2916vs, 2848s, 1760m, 1701s, 1463m, 1428m, 1411m, 1302m, 1276m, 1247m, 1220m, 1193m, 1168m, 1141m, 1115m, 938m, 720m.

¹H-NMR (400 MHz, CDCl₃, ppm, δ): 0.92 (t, 3H, CH₃); 1.26 (d, 6H, 2CH₃(i-Pr)); 1.30-1.47 (m, 14H, CH₂); 1.69 (m, 2H, CH₂); 1.80 (m, 2H, CH₂); 2.15 (s, 3H, CH₃); 2.59 (t, 2H, CH₂); 2.89 (m, 1H, CH(i-Pr)); 6.87 (d, 1H, CH(Ph)); 7.03 (dd, 1H, CH(Ph)); 7.16 (dd, 1H, CH(Ph)).

MS (m/z, CI): 332 [C₂₂H₃₆O₂]⁺; 150 [C₁₀H₁₄O]⁺.

### Example 7

### Thymol Laurate (IB570)

Thymol (2 g, 0.013 mol), lauric acid (5.33 g, 0.026 mol) and an 85% aqueous solution of phosphoric acid (3 drops) are mixed under magnetic stirring at 150° C. in vacuum for 12 hours. The crude is poured in chloroform (4 ml) and purified with a flash chromatographic column (silica gel, n-hexane 100%) thus yielding a colourless oil (1.2 g, 28%).

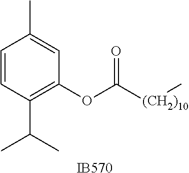

IR (cm⁻¹): 2957m, 2923s, 2853m, 1709s, 1620w, 1584w, 1505w, 1456m, 1416m, 1378w, 1363w, 1290w, 1226m, 1150s, 1111m, 1087m, 1058w, 946m, 814m, 805m, 721w.

¹H-NMR (400 MHz, CDCl₃, ppm, δ): 0.91 (t, 3H, CH₃); 1.22 (d, 6H, 2CH₃(i-Pr)); 1.30-1.46 (m, 16H, CH₂); 1.82 (m, 2H, CH₂); 2.34 (s, 3H, CH₃); 2.60 (t, 2H, CH₂); 2.98 (m, 1H, CH(i-Pr)); 6.82 (d, 1H, CH(Ph)); 7.04 (dd, 1H, CH(Ph)); 7.21 (dd, 1H, CH(Ph)).

MS (m/z, CI): 332 [C₂₂H₃₆O₂]⁺; 150 [C₁₀H₁₄O]+.

### Example 8

### Menthol Laurate (IB579)

Menthol (3 g, 0.019 mol), lauric acid (7 g, 0.35 mol) and an 85% aqueous solution of phosphoric acid (3 drops) are mixed under magnetic stirring at 100° C. under vacuum for 12 hours. The crude was poured in chloroform (4 ml) and purified with a flash chromatographic column (hexane 100%) yielding a colorless oil (1.6 g, 36%).

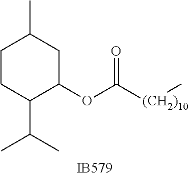

IR (cm⁻¹): 2956m, 2922s, 2853m, 1731s, 1683m, 1635m, 1558w, 1456m, 1369w, 1248w, 1175m, 1149m, 1107w, 1012m, 983m.

¹H-NMR (400 MHz, CDCl₃, ppm, δ): 0.78 (d, 6H, 2CH₃(i-Pr)); 0.86-1.09 (m, 10H, CH); 1.28-1.31 (m, 16H, CH₂); 1.51 (m, 1H, CH); 1.61-1.72 (m, 4H, CH), 1.88 (m, 1H, CH(i-Pr)); 2.00 (m, 1H, CH); 2.29 (t, 2H, CH₂), 4.70 (td, 1H, CHO).

ESI-MS (m/z): 361 [C₂₂H₄₂O₂Na]⁺.

### Example 9

### Evaluation of the Minimum Inhibitory Concentration (MIC) and Minimum Concentration Bactericidal (MBC) of the Compounds of the Invention Against Bacterial Strains of Avian Origin

### Materials and Methods:

Bacterial strains: the concentration of each bacterial strain was in the order of 10⁶ cfu/ml.
Determination of MIC: the test was carried out in a liquid culture medium on 96-well Microtiter plates.

Each compound was tested in triplicate and at the following concentrations: 5%, 2.5%, 1.25%, 0.625%, 0.312%, 0.16%, 0.08%.

The plates were incubated at 37° C. for 24 hours (in anaerobiosis for C. perfringens). After the incubation period, for each test, the lowest concentration was read being able to inhibit the growth of the microorganism.
Determination of MBC: the test was performed by withdrawing the contents of the wells corresponding to the value of MIC and the higher concentrations and putting such content in plates of culture specific for microorganisms tested. After the incubation period, for each test, the lowest concentration was read at which no bacterial growth was observed.

### Results:

RESULTS/MIC (%)

| Bacterial Strain | IB536 | IB540 | IB546 | IB574 | IB575 | IB559 | IB570 | IB579 | Thymol | Carvacrol |
|---|---|---|---|---|---|---|---|---|---|---|
| Escherichia coli | >10 | 0.75 | 5 | 0.75 | 2.5 | ≤0.093 | >10 | >10 | ≤0.093 | ≤0.093 |
| Salmonella | >10 | 0.75 | 1.25 | 0.375 | 5 | ≤0.093 | >10 | >10 | ≤0.093 | 0.375 |
| Typhimurium | | | | | | | | | | |
| Clostridium | >10 | 0.375 | 0.375 | 0.375 | 1.25 | ≤0.093 | >10 | >10 | ≤0.093 | ≤0.093 |
| perfringens | | | | | | | | | | |
| Salmonella | — | 0.312 | 1.25 | 0.625 | — | — | — | — | — | — |
| Enteritidis | | | | | | | | | | |

RESULTS/MBC (%)

| Bacterial Strain | IB536 | IB540 | IB546 | IB574 | IB575 | IB559 | IB570 | IB579 | Thymol | Carvacrol |
|---|---|---|---|---|---|---|---|---|---|---|
| Escherichia coli | >10 | 0.75 | 5 | 0.75 | 2.5 | ≤0.093 | >10 | >10 | ≤0.093 | ≤0.093 |
| Salmonella | >10 | 0.75 | 1.25 | 0.375 | 5 | ≤0.093 | >10 | >10 | ≤0.093 | 0.375 |
| Typhimurium | | | | | | | | | | |
| Clostridium | >10 | 0.75 | 0.75 | 0.375 | 2.5 | ≤0.093 | >10 | >10 | ≤0.093 | ≤0.093 |
| perfringens | | | | | | | | | | |
| Salmonella | — | 0.312 | 1.25 | 0.625 | — | — | — | — | — | — |
| Enteritidis | | | | | | | | | | |

Without wishing to be bound by any theory, it is supposed that the lower activity shown by some esters in in vitro tests could be ascribable to the presence of the ester moiety, which is believed to act as a protective group. In fact, the esterification protects the essential oils throughout the production process and handling of the food products containing the same, up to the first digestion step. Then, once the intestine area is reached, the lipases intervene on the ester molecules hydrolysing the ester bond and thus releasing the essential oils. In this way, the latter are free to exert their antibacterial activity in the intestine, i.e. exactly where pathogens such as E. coli and Salmonella are typically present.

The possibility to release essential oils once the esters of the invention contact lipases in the intestine area, i.e. the possibility to use the esters of the invention like pro-drug molecules, allows to achieve a number of advantages with respect to the essential oils as such. Said advantages mainly are:
higher yields during the production process due to the lower volatility of the esters,
less intense odour resulting in a higher palatability;
higher safety during the production process and handling due to the higher flash points of esters;
reduced reactivity towards other active principles present in food products, such as vitamins, metal oxides, etc . . . ;
combined action of the released essential oil and the carboxylic or dicarboxylic acid, the latter having itself antibacterial properties.

It has been observed also that essential oils and sulfonates of the invention have different solubility in water and melting points:

| | Solubility in water | Melting point | |
|---|---|---|---|
| Carvacrol | INSOLUBLE | 1° C. (boiling point 238° C.) | |
| IB546 | ~250 g/l at 25° C. | >270° C. (decomposed) | |
| Thymol | INSOLUBLE | 49-51° | C. |
| IB575 | ~190 g/l at 25° C. | 220° | C. |

The remarkable solubility of the compounds of the invention in water, compared to the insolubility of essential oils as such, is a matter of great interest. For example, these esters can be supplied also as beverage to animals. Indeed, by increasing the hydrophilicity of these products, also the bioavailability should improve.

Moreover, the higher melting points and the solid state (such as powders) instead of liquid oils ensure better handling in the production process, whereas higher flash points (lower flammability) result in more safety working conditions. Lower volatility is also an additional quality of the esters in terms of a reduced olfactory impact on animals during nourishment and a smaller loss ratio of active ingredient in manufacturing the food products.

1. A compound of formula A-O—R, wherein A-O— is a moiety of an essential oil active ingredient, said active ingredient having a functional group —OH, and R is a moiety of formula R₁(CO)— or formula A-O—(CO)—R₂—(CO)—, where R₁ is a saturated or unsaturated alkyl chain C4-C22 and R₂ is a saturated or unsaturated alkyl chain C1-C6.

2. The compound of claim 1, wherein said active ingredient is selected from the group consisting of menthol, thymol, eugenol, geraniol, vanillin, carvacrol, nerolidol, ethyl maltol, furaneol, 1-hexanol, 3-hexen-1-ol, linalool, alpha-santalol, nerol, terpineol, borneol, farnesol, citronellol, myrtenol, coumaric acid, para-hydroxybenzoic acid, gallic acid, protocatechuic acid, syringic acid, gentisic acid, caffeic acid, ferulic acid, sinapic acid, methyl salicylate, and mixtures thereof.

3. The compound of claim 2, wherein said active ingredient is selected from the group consisting of menthol, thymol, eugenol, vanillin, carvacrol, and mixtures thereof.

4. The compound of claim 1, wherein said moiety R has formula R₁(CO)— and R₁ is a saturated alkyl chain C8-C18.

5. The compound of claim 1, wherein said moiety R has formula A-O—(CO)—R₂—(CO)— and R₂ [è] is a saturated or mono-unsaturated alkyl chain C1-C4.

6. A process for the preparation of the compound of formula A-O—R of claim 1, wherein said moiety R has formula R₁(CO)—, said process comprising the steps of:
a) providing a carboxylic acid of formula R₁(CO)OH and an essential oil active ingredient of formula A-OH, being the carboxylic acid in excess over the active ingredient; and
b) heating up to melting of both the components of step a), under vacuum, under stirring and in the presence of an acid catalyst, and reacting for at least 9 hours.

7. A process for the preparation of the compound of formula A-O—R of claim 1, wherein said moiety R has formula A-O—(CO)—R₂—(CO)—, said process comprising the steps of:
a) reacting a dicarboxylic acid of formula HO(CO)—R₂—(CO)OH with oxalyl chloride under dry conditions, thus obtaining the respective acyl chloride; and
b) reacting said acyl chloride with an essential oil active ingredient of formula A-OH.

8. The process of claim 6, wherein said an essential oil active ingredient of formula A-OH is preliminarily reacted with sulfuric acid to give the respective sulfonate.

9. A sulfonate of menthol, thymol, eugenol, geraniol, vanillin, carvacrol, nerolidol, ethyl maltol, furaneol, 1-hexanol, 3-hexen-1-ol, linalool, alpha-santalol, nerol, terpineol, borneol, farnesol, citronellol, myrtenol, coumaric acid, para-hydroxybenzoic acid, gallic acid, protocatechuic acid, syringic acid, gentisic acid, caffeic acid, ferulic acid, sinapic acid, or methyl salicylate, or a salt thereof.

10. A human or animal food product, comprising the compound of formula A-O—R of claim 1 as a food ingredient.

11. The compound of claim 2, wherein said moiety R has formula R₁(CO)— and R₁ is a saturated alkyl chain C8-C18.

12. The compound of claim 3, wherein said moiety R has formula R₁(CO)— and R₁ is a saturated alkyl chain C8-C18.

13. The compound of claim 2, wherein said moiety R has formula A-O—(CO)—R₂—(CO)— and R₂ is a saturated or mono-unsaturated alkyl chain C1-C4.

14. The compound of claim 3, wherein said moiety R has formula A-O—(CO)—R₂—(CO)— and R₂ is a saturated or mono-unsaturated alkyl chain C1-C4.

15. The process of claim 7, wherein said an essential oil active ingredient of formula A-OH is preliminarily reacted with sulfuric acid to give the respective sulfonate.

16. A human or animal food product, comprising the sulfonate of claim 9, as a food ingredient.
